(19) United States
(12) Patent Application Publication
(10) Pub. No.: US 2016/0175522 A1
(43) Pub. Date: Jun. 23, 2016

(54) CLOSED LOOP ANESTHETIC DELIVERY

(71) Applicant: Ethicon Endo-Surgery, Inc., Cincinnati, OH (US)

(72) Inventor: James F. Martin, Lebanon, OH (US)

(21) Appl. No.: 15/054,658

(22) Filed: Feb. 26, 2016

Related U.S. Application Data
(63) Continuation of application No. 12/349,872, filed on Jan. 7, 2009.
Application No. 12/349,872 is a continuation of application No. 10/886,322, filed on Jul. 7, 2004, now abandoned.

(51) Int. Cl. A61M 5/172; A61B 5/00

(52) U.S. Cl. CPC A61M 5/1723; A61B 5/4821; A61B 5/4839

(57) ABSTRACT

A method for delivering a sedation drug comprising administering a drug to a patient while requesting the patient to respond to an instruction, monitoring a patient's BIS values, bringing the patient to a level of anesthesia where the patient fails to respond to the request within a predetermined response time, and determining a BIS value that coincides with the level of anesthesia corresponding to the failure to respond.

### RELATED APPLICATION

This is a continuation of application Ser. No. 12/349,872, filed on Jan. 7, 2009, which is a continuation of application Ser. No. 10/886,322, filed on Jul. 7, 2004, which is incorporated herein by reference, in its entirety.

### FIELD OF THE INVENTION

The present invention relates to sedation drug delivery and, more particularly, closed-loop sedation drug delivery.

### BACKGROUND OF THE INVENTION

Various automated delivery systems have been proposed for the administration of drugs such as anesthetics, sedatives and analgesics for achieving anesthesia. These systems range from “open-loop” systems, relying on pharmacokinetic models of the anesthetic drug to control delivery, to “closed-loop” systems, relying on measures of the depth of anesthesia to control delivery. The term “anesthesia,” as used herein, refers to the continuum of hypnosis and analgesia achieved via sedation drugs, and ranges from anxiolysis to general anesthesia. The term “sedation drug,” as used herein, refers to the class of drugs employed by anesthesiologists in inducing sedation or anesthesia, and includes hypnotics, analgesics and the like.

One “closed-loop” system, described in Absalom, A., Sutcliffe, N., and Kenny G., “Closed-loop control of anesthesia using Bispectral index: performance assessment in patients undergoing major orthopedic surgery under combined general and regional anesthesia”, Anesthesiology, Vol. 96(1), pp 67-73, January 2002, uses the Bispectral Index (BIS), which is a continuously processed EEG parameter that measures the state of brain function during administration of sedation drugs, as the measure of depth of anesthesia. BIS is a quantitative EEG analysis technique that has been developed for use during anesthesia. Bispectral analysis of EEG measures consistency of phase and power relationships among the various frequencies of the EEG. The index is derived from both a power spectral analysis and a time domain analysis.

Although BIS provides decent population sedation and anesthesia values, there is significant patient-to-patient variability. The BIS index is a number between 0 and 100 scaled to correlate with important clinical end points during administration of sedation drugs. A value of 100 represents an awake clinical state while 0 denotes an isoelectric EEG. At a BIS value of 60 the patient typically has a very low probability of consciousness. BIS values are inversely proportional to the plasma level of concentration of drugs in the patient, i.e. the lower the BIS value, the higher the concentration of drugs in the patient and the higher the BIS value, the lesser the concentration of drugs in the patient; however, each BIS spectrum varies significantly from patient to patient. As a result, the use of a model BIS spectrum to assess the depth of anesthesia is not reliable in individual patients. Accordingly, there is a need to tune BIS to each patient individually in order to correlate and assess the depth of anesthesia of the patient and thereby “close the loop” on the sedation drug delivery system.

Therefore, in 3 separate studies (See Leslie, K., Absalonm, A., and Kenny, G., “Closed loop control of sedation for colonoscopy using Bispectral Index”, Anesthesia, Vol. 57(7), pp. 693-697, July 2002; Absalom, A., Sutcliffe, N., and Kenny G., “Closed-loop control of anesthesia using Bispectral index: performance assessment in patients undergoing major orthopedic surgery under combined general and regional anesthesia”, Anesthesiology, Vol. 96(1), pp. 67-73, January 2002; and Absalom, A. and Kenny, G., “Closed-loop control of propofol anesthesia using Bispectral index: performance assessment in patients receiving computer-controlled propofol and manually controlled remifentanil infusions for minor surgery”, Br. J. Anaesthesia, Vol. 90(6), pp. 737-741, June 2003) the patient's individual BIS values have been correlated with the individual's level of sedation first, using manual titration of sedation. Then, based on the manually obtained BIS values, a setpoint BIS value was determined and closed-loop control was initiated. This procedure is feasible only in a research setting and would be unacceptable in a clinical setting since the correlation of individual BIS values to the individual's level of anesthesia is time-consuming. Accordingly, it would be desirable to provide a method for efficiently tuning BIS to an individual patient in an operational setting. S. D. Kelly, Monitoring Level of Consciousness During Anesthesia and Sedation, provides a detailed explanation of BIS and how it works and is available online at http://www.aspectmedical.com.

### SUMMARY OF THE INVENTION

A first embodiment of the present invention provides a method for delivering a sedation drug comprising the steps of: administering a sedation drug to a patient while requesting the patient to respond to an instruction; monitoring a patient's BIS values; bringing the patient to a level of anesthesia where the patient fails to respond or slowly responds to the request; determining a BIS value that coincides with the level of anesthesia at which the patient fails to respond or slowly responds to the request; and establishing a BIS setpoint. Closed-loop delivery of the sedation drug is initiated to maintain the patient's BIS value at the setpoint.

A second embodiment of the present invention provides a drug delivery apparatus having an automated response monitoring system (ARM), a Bispectral Index (BIS) monitoring apparatus to monitor a patient's BIS values during delivery of a sedation drug, and a sedation drug infusion device.

Other embodiments, objects, features and advantages of the present invention will become apparent to those skilled in the art from the detailed description, the accompanying drawings and the appended claims.

### BRIEF DESCRIPTION OF THE DRAWINGS

FIG. 1 is a block diagram showing a sedation drug delivery system in accordance with one embodiment of the present invention;

FIG. 2 is a flow chart showing one method in accordance with the present invention; and

FIG. 3 is a diagram of an automated response monitoring (ARM) system.

### DETAILED DESCRIPTION OF THE INVENTION

One embodiment of the present invention provides a closed-loop sedation drug delivery system by combining the features of BIS with the patient specific features of an Automated Response Monitoring system (ARM) to calibrate a set point and thereby “close-the-loop” on sedation drug delivery. Alternatively, other systems for indexing of depth of anesthesia may be substituted for BIS according to the present invention, such as, for example, Narcotrend and various audio evoked potential (AEP) devices.

ARM by itself is a binary measure of responsiveness (i.e. the patient either responds or does not respond). ARM can play an integral role in a sedation drug delivery system by identifying the transition from moderate to deep sedation. However, since it is a binary measure it cannot provide adequate information regarding the patient's depth of anesthesia following loss of responsiveness. Because the patient loses responsiveness, ARM alone cannot be used to provide a closed loop sedation drug delivery system. Nevertheless, ARM can be used in conjunction with BIS (or other indices of depth of anesthesia) to efficiently determine the patient's level of anesthesia and “close-the-loop” on sedation.

BIS has been used to measure changes in the effects of sedation drugs, such as anesthetics and the like, on the brain and, more specifically, the hypnotic state of the patient. BIS monitors are available commercially from Aspect Medical Systems, 141 Needham St., Newton, Mass. 02464. When a patient is more sedated, BIS values are lower and when a patient is less sedated, BIS values are higher. A patient's BIS values reflect the patient's reaction to a drug. A more sensitive patient will display a greater decrease in BIS values than a less sensitive patient when administered the same dosage of a drug. Thus, BIS can measure a patient's relative sedation level; however, the wide variability of patient sensitivity to drugs, even among patients having similar physical attributes, precludes the use of BIS alone to determine a patient's level of anesthesia. Thus, generally, it is not feasible to produce a general population BIS model that correlates a BIS range to an individual's level of anesthesia. BIS should be correlated with the individual patient to determine the patient's level of anesthesia. This can be achieved by correlating the patient's responses to ARM with the patient's individual BIS values to more precisely determine the patient's level of anesthesia and further, to help establish a set-point or target level of anesthesia for the patient.

The use of ARM to assess a patient's level of anesthesia is described in U.S. patent application Ser. No. 10/674,160, filed Sep. 29, 2003, which is hereby incorporated by reference. As described in the application, several methods and apparatuses may be used to monitor a patient's level of anesthesia using ARM. In sum, ARM is a patient response system that sends various requests to a patient to receive a patient's response and then analyzes the patient's responses to the requests. By analyzing the patient's responses, the patient's level of anesthesia can be determined. The patient may also reach a level of anesthesia where the patient is no longer responsive to ARM or the patient fails to respond within a predetermined period of time. Several different criteria may be used in determining the end point when a patient is considered to have lost responsiveness to ARM. For example, as discussed in the aforementioned application, loss of ARM may occur when a patient fails to respond within a certain period of time after a request has been sent to the patient. Loss of ARM may also occur when the patient's response does not meet a minimum threshold response level. Thus, the clinician may determine the point at which the patient loses responsiveness to ARM. Although the criteria for what determines loss of ARM could be chosen by the clinician, the point at which the patient is deemed to have lost responsiveness to ARM is always correlated to the patient's BIS values for that specific point. By doing so, BIS values are correlated to the individual patient.

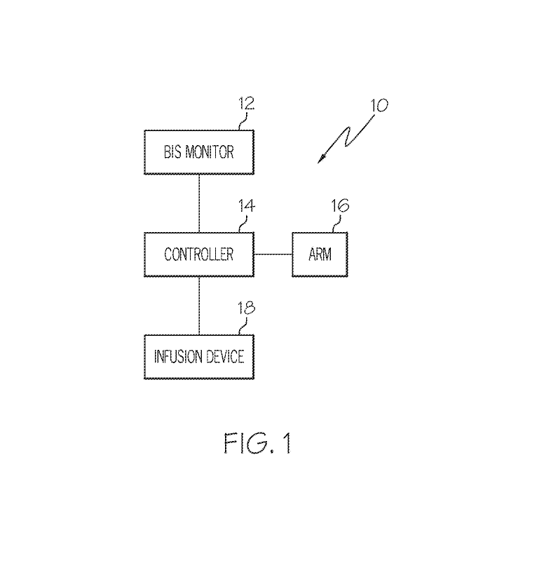

FIG. 1 is a block diagram of a sedation drug delivery system 10 in accordance with one embodiment of the present invention. The system 10 includes a BIS monitor 12, a controller 14, an ARM system 16 and an infusion device 18. The infusion device 18 can be an automated infusion pump that is controlled via the controller 14. The term “controller” as used herein includes a single logic device that performs the disclosed function as well as any combination of logic devices that perform the disclosed functions. In accordance with one embodiment of the present invention, the controller 14 evaluates the output from the BIS monitor 12 and instructs the infusion device 18 to continue to deliver the sedation drug based on the output from the BIS monitor 12 and its relationship to a BIS setpoint established via the ARM system 16.

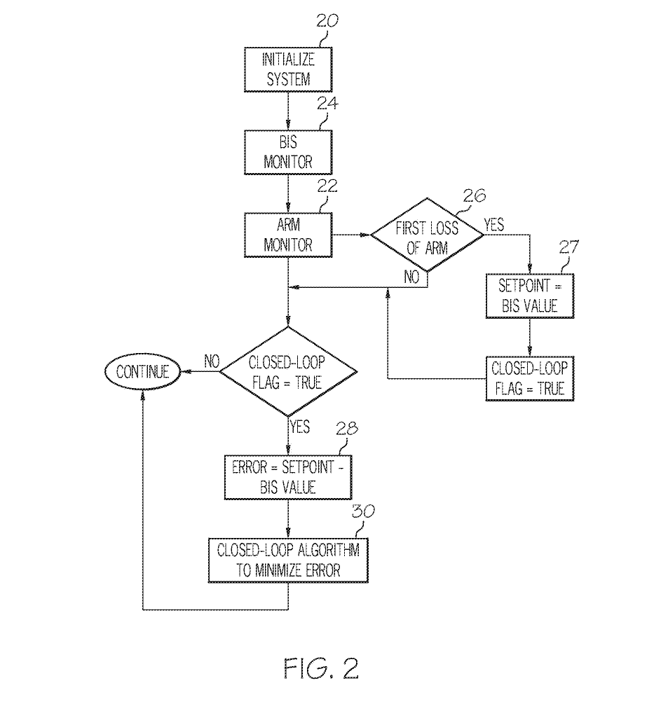

A method in accordance with one embodiment of the present invention is diagrammed as a flow chart in FIG. 2. To begin sedation, in step 20 the clinician initializes the system by programming the controller 14 with information relevant to the patient, such as name, age and weight, etc. Based upon the input, the controller 14 will select or calculate an infusion modality or rate for the patient or the clinician can set a drug infusion rate. One example of a method whereby the controller 14 establishes the infusion rate based on a loading dose is described in commonly assigned U.S. patent application Ser. No. ______ entitled “Dosage Control For Drug Delivery System” (attorney docket number 451231-00049), filed herewith.

With the initialization of the system by the clinician, as show in step 20, the infusion device 18 starts delivering the identification infusion rate and the controller 14 signals the BIS monitor 12 to begin monitoring the BIS index for the patient in step 24 and also signals the ARM system 16 to begin requesting responses from the patient in step 22. In step 26, the ARM system monitors the patient for responses to its requests. The device stays in an “open-loop,” delivering the selected identification infusion rate and monitoring BIS and ARM, until the patient loses ARM response by either failing to respond to a predetermined number of requests (e.g., 1 to 3), or failing to respond within a predetermined response time (e.g., a predetermined number of seconds). The ARM system then signals the controller 14 of the loss of responsiveness to ARM and the device switches to “closed-loop” mode, adjusting the infusion rate, in an attempt to minimize the error (i.e., the difference between the Setpoint and the measured BIS value). When the device is in closed-loop mode, various known closed-loop algorithms may be used.

The controller 14 receives BIS values from the BIS monitor 12 and uses the patient's BIS index at that point where responsiveness to ARM was lost as a setpoint (see step 27) based upon which the controller 14 monitors further drug infusion in step 28. The setpoint may not be based on the BIS index at that point itself but, depending upon the nature of the surgical procedure, may be based on a BIS value that is offset from it. For example, if the procedure is one that does not require deep anesthesia, the setpoint may be set several points higher than the point at which the patient lost responsiveness to ARM. Likewise, if the procedure is one that requires much deeper anesthesia (e.g., general anesthesia), the setpoint may be set several points lower than the point at which the patient lost response to ARM.

With the BIS setpoint established, the controller 14 generates an error between the output from the BIS monitor 12 and the BIS setpoint (see step 28). The error is then minimized in step 30 using a closed-loop algorithm. The action of the closed-loop algorithm may depend on the sedation drug, the nature of the procedure, and the patient's characteristics. For example, if the patient's BIS index is substantially greater than the setpoint, the controller may increase the infusion rate. On the other hand, if the patient's BIS is substantially less than the setpoint, the controller may stop (or slow) the drug infusion. The invention also is not limited to infusion rate control based solely upon BIS monitoring but rather is open to systems in which either the BIS index comparison or the response to ARM or both are used.

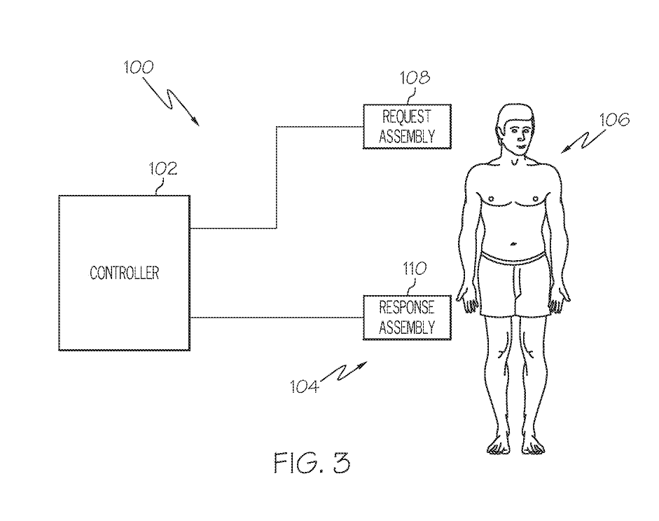

An example of how ARM works is shown in the drawings. FIG. 3 illustrates a conscious sedation system 100 including a controller 102 and a response testing apparatus 104. The controller 102 generates a request for a predetermined response from a patient 106 and analyzes at least a response generated by the patient 106 to the request to determine a level of sedation of the patient 106. The response testing apparatus 104 includes a request assembly 108 and a response assembly 110. The request assembly 108 communicates to the patient 106 the request generated by the controller 102. The response assembly 110 is used by the patient 106 to generate the response and communicates the response to the controller 102. Examples of response assemblies particularly useful herein are hand grip assemblies as described in detail in commonly assigned U.S. patent application Ser. No. 10/674,160 entitled “Response Testing for Conscious Sedation Involving Hand Grip Dynamics,” filed Sep. 29, 2003. The response assembly includes a handpiece which senses a dynamic variable of a hand grip response made by the patient to the request and communicates the dynamic variable to the controller which analyzes at least the dynamic variable to determine a level of anesthesia of the patient.

The method of using ARM comprises applying a stimuli or request for a predetermined response to the patient; instructing the patient to respond to the stimuli; monitoring the patient's response to the stimuli; and repeating the steps until patient loses responsiveness to ARM. In the meantime, the patient's individual BIS values associated with the patient's level of anesthesia are also monitored. The BIS value at which the patient loses responsiveness to ARM is recorded and used to calibrate BIS to the individual patient. In the preferred embodiment, the BIS value at which the patient loses responsiveness to ARM is used as the BIS setpoint at which to maintain the patient's level of anesthesia. Nevertheless, the patient's BIS setpoint may be increased or decreased according to the physician's discretion. It is often a goal of a medication delivery system to achieve and maintain a desired effect on the patient. This desired effect or level of effect is referred to as the setpoint. The set point specified by the anesthetist or other health care professional is preferably approached and maintained as closely as possible during the maintenance of the anesthesia.

By integrating the ARM system described above with the features associated with BIS, the BIS can be tuned to the individual patient, and set values can be established thereby closing the loop on the sedation drug delivery system.

In one embodiment of the invention, a drug is administered to the patient until loss of ARM. This may be accomplished by gradually increasing the infusion rate. For example, the system gradually increases the drug infusion rate, starting at 50 μg/kg/min and stepping up the rate 25 μg/kg/min every 60 seconds until the patient loses responsiveness to ARM over three consecutive samples (i.e., the patient fails to respond to three consecutive ARM requests). At this point, the average BIS value over the three consecutive samples is used as the setpoint for the closed-loop controller. This BIS value (i.e., the BIS setpoint) corresponds to the target level of anesthesia at which the patient should be maintained during the procedure.

The infusion rate may be operated with various profiles in bringing the patient to loss of ARM. Similarly, different end points can be used to define the loss of ARM depending upon the age, health, and other characteristics of the patient. For example, the infusion pump can increase infusion rate at a constant rate or a constant slope ramp. It could also be a variable slope ramp or start high and have a negative slope ramp as long as the patient is taken to loss of ARM safely and quickly, preferably within five minutes. Once the BIS values are determined over the range at which the patient loses responsiveness to ARM, a BIS setpoint is established and the sedation drug delivery system maintains the desired level of anesthesia at the BIS setpoint for the remainder of the procedure. If the clinician wants a different level of anesthesia later in the procedure, he can accomplish this by changing the BIS setpoint value. For example, if the clinician wants a deeper level of anesthesia for a more sensitive aspect of the procedure, the clinician may lower the BIS set point. However, instead of blindly setting a population BIS value to be the setpoint, the user will be adjusting a BIS value that has been tuned to the specific patient via ARM. Accordingly, the clinician can close the loop on the sedation drug delivery system through this integration of the patient's response to ARM and the patient's BIS values. Whereas, previously, with ARM alone, it was not possible to determine the patient's depth of anesthesia, however, by keeping the patient's level of anesthesia at or near the BIS value at which the patient lost responsiveness to ARM, over-sedation is prevented.

A second embodiment of the present invention provides a drug delivery apparatus having an automated response monitoring system (ARM), a Bispectral Index (BIS) monitoring apparatus to monitor a patient's BIS values during delivery of a sedation drug, and a sedation drug infusion device.

Although the invention is shown and described with respect to certain embodiments, particularly, embodiments utilizing BIS as an index of the depth of anesthesia, it is obvious that equivalents and modifications will occur to those skilled in the art upon reading and understanding the specification and the appended claims. The present invention includes all such equivalents and modifications and is limited only by the scope of the claims. For example, any device that provides an index of depth of anesthesia may be substituted for BIS, including, but not limited to, Narcotrend and various AEP devices.

All documents cited are, in relevant part, incorporated herein by reference. The citation of any document is not to be construed as an admission that it is prior art with respect to the present invention.

What is claimed:

1. A method for delivering a sedation drug comprising the steps of:
providing at least one device for infusing a sedation drug and for measuring a patient's index of depth of anesthesia;
initially administering a sedation drug to a patient in an open loop mode while requesting the patient to respond to an instruction;
monitoring a patient's index of depth of anesthesia;
bringing the patient to a level of anesthesia where the patient fails to respond to the request within a predetermined response time;
determining an index of depth of anesthesia value that coincides with the level of anesthesia where the patient fails to respond; and
adjusting the administration of the sedation drug to the patient to a closed loop mode.

2. The method of claim 1 wherein the request is generated by an automated response monitoring system (ARM).

3. The method of claim 2 wherein the patient is brought to a level of anesthesia at which the patient does not respond to ARM.

4. The method of claim 3 wherein ARM includes:
a controller which generates a request for a predetermined response from the patient and which analyses a response generated by the patient to the request for a predetermined response; and
a response testing apparatus including:
a request assembly which communicates to the patient the request generated by the controller; and
a response assembly which is used by the patient to generate the response and which communicates the response to the controller,
wherein at least one of the request assembly and the response assembly communicates at least one of the request and the response between the controller and the patient.

5. The method of claim 2 further comprising the step of establishing the index of depth of anesthesia value that coincides with the level of anesthesia where the patient fails to respond as an index of depth of anesthesia setpoint and continuing to administer the drug to the patient while monitoring the patient's index of depth of anesthesia values relative to the index of depth of anesthesia setpoint.

6. The method of claim 5 further comprising the step of changing a patient's level of anesthesia by changing the index of depth of anesthesia setpoint relative to the index of depth of anesthesia value that coincides with the level of anesthesia where the patient fails to respond.

7. The method of claim 1 wherein the step of initially administering the drug to the patient includes infusing the drug with an infusion rate profile in which the infusion rate is gradually increasing.

8. The method of claim 1 wherein the step of initially administering the drug to the patient includes infusing the drug with an infusion rate profile in which the infusion rate is gradually decreasing.

9. The method of claim 1 wherein the step of initially administering the drug to the patient includes infusing the drug with an infusion rate profile in which the infusion rate is constant.

10. The method of claim 1 wherein the step of initially administering the drug to the patient includes infusing the drug with an infusion rate profile in which the infusion rate increases at a constant rate.

11. The method of claim 1 wherein the step of initially administering the drug to the patient includes infusing the drug with an infusion rate profile in which the infusion rate increases at a non-constant rate.

12. The method of claim 6 wherein the step of continuing to administer the drug is adjusted based on a difference between the patient's index of depth of anesthesia value and the index of depth of anesthesia setpoint.

13. The method of claim 5 wherein the step of continuing to administer the drug is conducted in a closed loop with the step of monitoring the patient's index of depth of anesthesia values relative to the index of depth of anesthesia setpoint.

14. The method of claim 1 wherein said index of depth of anesthesia is the Bispectral Index.

15. The method of claim 1 wherein said index of depth of anesthesia is based on audio evoked potential.

16. The method of claim 1 wherein said index of depth of anesthesia is Narcotrend.
